(19) United States
(12) Patent Application Publication
(10) Pub. No.: US 2018/0117005 A1
(43) Pub. Date: May 3, 2018

(54) COMPOSITIONS FOR THE TREATMENT OF CANCER

(71) Applicant: THE GENERAL HOSPITAL CORPORATION, Boston, MA (US)

(72) Inventors: Patrick Reeves, Boston, MA (US); Mark C. Poznansky, Boston, MA (US)

(21) Appl. No.: 15/568,960

(22) PCT Filed: Apr. 25, 2016
(86) PCT No.: PCT/US16/29258

§ 371 Date: Oct. 24, 2017

Related U.S. Application Data
(60) Provisional application No. 62/152,815, filed on Apr. 24, 2015.
(60) Provisional application No. 62/152,824, filed on Apr. 25, 2015.

(51) Int. Cl. A61K 31/395; A61K 45/06; A61P 35/00; A61P 37/04

(52) U.S. Cl. CPC A61K 31/395; A61P 37/04; A61P 35/00; A61K 45/06

(57) ABSTRACT

The invention described herein relates to compositions for treating cancer in a patient, or a tumor cell, by administering an effective amount of AMD3100 over a period of time.

### BACKGROUND OF THE INVENTION

Cell movement in response to specific stimuli is observed to occur in prokaryotes and eukaryotes. Cell movement seen in these organisms has been classified into three types: chemotaxis or the movement of cells along a gradient towards an increasing concentration of a chemical; negative chemotaxis which has been defined as the movement down a gradient of a chemical stimulus; and chemokinesis or the increased random movement of cells induced by a chemical agent.

Chemotaxis and chemokinesis have been observed to occur in mammalian cells in response to the class of proteins, called chemokines. Additionally, chemorepellent, or fugetactic, activity has been observed in mammalian cells. For example, some tumor cells secrete concentrations of chemokines that are sufficient to repel immune cells from the site of a tumor, thereby reducing the immune system's ability to target and eradicate the tumor. Metastasizing cancer cells may use a similar mechanism to evade the immune system.

Anti-fugetactic agents have been described that inhibit the fugetactic activity of tumor cells and allow the patient's immune system to target the tumor (see US 2008/0300165, incorporated herein by reference in its entirety). However, treatment with such agents may not be sufficient to eradicate a tumor in all patients, depending on the type of tumor, size of tumor, number of metastases, site(s) of metastasis, patient's health, etc.

There remains a need for treatments and compositions that target tumors to efficiently kill tumors and/or metastasizing cancer cells.

### SUMMARY OF THE INVENTION

This invention relates to an aggregate unit dose of AMD3100 that inhibits the level of fugetaxis in a solid tumor. Specifically, this invention provides for an aggregate unit dose of AMD3100 that provides for therapeutic window to treat a tumor with conventional therapies while the tumor is susceptible to such therapy as a result of said aggregate unit dose.

Accordingly, in one embodiment there is provided an aggregate unit dose of AMD3100 that ranges from 50 to 350 mg/kg over a 7 day period of time with the understanding that said unit dose can be administered over a shorter period of time (e.g., 5 days) or a longer period of time (e.g., 14 days) depending upon the amount of said aggregate unit dose, the age, weight and condition of the patient as well as the rate of inhibition of fugetaxis by the tumor.

Use of this aggregate unit dose allows for attenuation of the fugetactic effect of AMD3100 which restore in whole or in part the endogenous immune defenses against the tumor, and also allow anti-cancer agents (e.g., chemotherapeutic agents, immunotherapeutic agents, radiotherapeutic agents, and the like) to better access the tumor in order to reduce or eradicate the tumor. Without being bound by theory, it is believed that the aggregate unit dose of AMD3100 allows for either co-administration or sequential administration of an anti-cancer therapy so as to improve the efficacy of the therapy.

Although anti-fugetactic agents alone have been suggested for cancer treatment, it is believed that combination therapy as described herein will result in more efficient tumor targeting and better patient outcomes. Without being bound by theory, it is believed that such methods are especially beneficial, by way of non-limiting example, if the tumor is large in size, there are multiple tumors in the patient, the patient's immune system is compromised, etc.

As many as 85% of solid tumors and leukemias express CXCL12 at a level sufficient to have fugetactic effects, e.g. repulsion of immune cells from the tumor. Cancers that express CXCL12 at such levels include, but are not limited to, prostate cancer, lung cancer, breast cancer, pancreatic cancer, ovarian cancer, gastric cancer, esophageal cancer, and leukemia.

One aspect of the invention relates to an aggregate unit dose that is effective at inhibiting fugetactic defenses around, by or of a tumor.

One aspect of the invention relates to use of an aggregate unit dose to increase migration of immune cells to a tumor site in a patient having a cancer. Optionally, the patient is administered at least one additional anti-cancer agent.

One aspect of the invention relates to use of an aggregate unit dose to kill a cancer cell. Optionally, the patient is administered at least one additional anti-cancer agent.

In one embodiment, the tumor is a solid tumor. In one embodiment, the tumor is a non-solid tumor. In one embodiment, the tumor is a leukemia.

In one embodiment, the at least one additional anti-cancer agent is a chemotherapeutic agent, a radiotherapy agent, and/or an anti-cancer vaccine.

Without being bound by theory, it is believed that the aggregate unit dose down regulates the fugetactic ability of the tumor, for a sufficient period of time to allow either the patient's own immune system and/or conventional therapies to effectively interface with the tumor.

The aggregate unit dose can be administered for or during a period of about 5 to about 14 days. It is understood that shorter or longer periods also are suitable.

### DETAILED DESCRIPTION

After reading this description, it will become apparent to one skilled in the art how to implement the invention in various alternative embodiments and alternative applications. However, not all embodiments of the present invention are described herein. It will be understood that the embodiments presented here are presented by way of an example only, and not limitation. As such, this detailed description of various alternative embodiments should not be construed to limit the scope or breadth of the present invention as set forth below.

Before the present invention is disclosed and described, it is to be understood that the aspects described below are not limited to specific compositions, methods of preparing such compositions, or uses thereof as such may, of course, vary. It is also to be understood that the terminology used herein is for the purpose of describing particular aspects only and is not intended to be limiting.

Definitions

Unless defined otherwise, all technical and scientific terms used herein have the same meaning as commonly understood by one of ordinary skill in the art to which this invention belongs.

In this specification and in the claims that follow, reference will be made to a number of terms that shall be defined to have the following meanings:

The terminology used herein is for the purpose of describing particular embodiments only and is not intended to be limiting of the invention. As used herein, the singular forms “a”, “an” and “the” are intended to include the plural forms as well, unless the context clearly indicates otherwise.

All numerical designations, e.g., pH, temperature, time, concentration, amounts, and molecular weight, including ranges, are approximations which are varied (+) or (−) by 10%, 1%, or 0.1%, where appropriate. It is to be understood, although not always explicitly stated, that all numerical designations may be preceded by the term “about.” It is also to be understood, although not always explicitly stated, that the reagents described herein are merely exemplary and that equivalents of such are known in the art.

“Optional” or “optionally” means that the subsequently described event or circumstance can or cannot occur, and that the description includes instances where the event or circumstance occurs and instances where it does not.

The term “comprising” or “comprises” is intended to mean that the compositions and methods include the recited elements, but not excluding others. “Consisting essentially of” when used to define compositions and methods, shall mean excluding other elements of any essential significance to the combination. For example, a composition consisting essentially of the elements as defined herein would not exclude other elements that do not materially affect the basic and novel characteristic(s) of the claimed invention. “Consisting of” shall mean excluding more than trace amount of other ingredients and substantial method steps recited. Embodiments defined by each of these transition terms are within the scope of this invention.

The terms “patient,” “subject,” “individual,” and the like are used interchangeably herein, and refer to any animal, or cells thereof whether in vitro or in situ, amenable to the methods described herein. In a preferred embodiment, the patient, subject, or individual is a mammal. In some embodiments, the mammal is a mouse, a rat, a guinea pig, a non-human primate, a dog, a cat, or a domesticated animal (e.g. horse, cow, pig, goat, sheep). In especially preferred embodiments, the patient, subject or individual is a human.

The term “treating” or “treatment” covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. For example, treatment of a cancer or tumor includes, but is not limited to, reduction in size of the tumor, elimination of the tumor and/or metastases thereof, inhibition of metastasis of the tumor, remission of the cancer, reduction or elimination of at least one symptom of the cancer, and the like.

The term “administering” or “administration” of an agent, drug, or a natural killer cell to a subject includes any route of introducing or delivering to a subject a compound to perform its intended function. Administration can be carried out by any suitable route, including orally, intranasally, parenterally (intravenously, intramuscularly, intraperitoneally, or subcutaneously), or topically. Administration includes self-administration and the administration by another.

It is also to be appreciated that the various modes of treatment or prevention of medical diseases and conditions as described are intended to mean “substantial,” which includes total but also less than total treatment or prevention, and wherein some biologically or medically relevant result is achieved.

The term “separate” administration refers to an administration of at least two active ingredients at the same time or substantially the same time by different routes.

The term “sequential” administration refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

The term “simultaneous” therapeutic use refers to the administration of at least two active ingredients by the same route and at the same time or at substantially the same time.

The term “therapeutic” as used herein means a treatment and/or prophylaxis. A therapeutic effect is obtained by suppression, remission, or eradication of a disease state.

The term “therapeutically effective amount” or “effective amount” refers to an amount of the agent that, when administered, is sufficient to cause the desired effect.

By “fugetactic activity” it is meant the ability of an agent to repel (or chemorepel) a eukaryotic cell with migratory capacity (i.e., a cell that can move away from a repellant stimulus). Accordingly, an agent with fugetactic activity is a “fugetactic agent.” Such activity can be detected using any of a variety of systems well known in the art (see, e.g., U.S. Pat. No. 5,514,555 and U.S. Patent Application Pub. No. 2008/0300165, each of which is incorporated by reference herein in its entirety). A preferred system for use herein is described in U.S. Pat. No. 6,448,054, which is incorporated herein by reference in its entirety.

The term “fugetactic effect” refers to the chemorepellant effect of a chemokine secreted by a cell, e.g. a tumor cell. Usually, the fugetactic effect is present in an area around the cell wherein the concentration of the chemokine is sufficient to provide the fugetactic effect. Some chemokines, including interleukin 8 and CXCL12, may exert fugetactic activity at high concentrations (e.g., over about 100 nM), whereas lower concentrations exhibit no fugetactic effect and may even be chemoattractant.

The term “anti-fugetactic effect” refers to the effect of an anti-fugetactic agent such as AMD3100 to attenuate or eliminate the fugetactic effect of the chemokine.

The term “anti-cancer therapy” as used herein refers to conventional cancer treatments, including chemotherapy and radiotherapy, as well as vaccine therapy.

AMD3100 (plerixafor; 1,1′-[1,4-Phenylenebis(methylene)bis [1,4,8,11-tetraazacyclotetradecane]). AMD3100 is described in U.S. Pat. No. 5,583,131, which is incorporated by reference herein in its entirety.

Chemotherapy Agents

In one aspect of the present invention, AMD3100 is administered in combination with a chemotherapy agent. The chemotherapy agent may be any agent having a therapeutic effect on one or more types of cancer. Many chemotherapy agents are currently known in the art. Types of chemotherapy drugs include, by way of non-limiting example, alkylating agents, antimetabolites, anti-tumor antibiotics, totpoisomerase inhibitors, mitotic inhibitors, corticosteroids, and the like.

Non-limiting examples of chemotherapy drugs include: nitrogen mustards, such as mechlorethamine (nitrogen mustard), chlorambucil, cyclophosphamide (Cytoxan®), ifosfamide, and melphalan); Nitrosoureas, such as streptozocin, carmustine (BCNU), and lomustine; alkyl sulfonates, such as busulfan; Triazines, such as dacarbazine (DTIC) and temozolomide (Temodar®); ethylenimines, such as thiotepa and altretamine (hexamethylmelamine); platinum drugs, such as cisplatin, carboplatin, and oxalaplatin; 5-fluorouracil (5-FU); 6-mercaptopurine (6-MP); Capecitabine (Xeloda®); Cytarabine (Ara-C®); Floxuridine; Fludarabine; Gemcitabine (Gemzar®); Hydroxyurea; Methotrexate; Pemetrexed (Alimta®); anthracyclines,such as Daunorubicin, Doxorubicin (Adriamycin®), Epirubicin, Idarubicin; Actinomycin-D; Bleomycin; Mitomycin-C; Mitoxantrone; Topotecan; Irinotecan (CPT-11); Etoposide (VP-16); Teniposide; Mitoxantrone; Taxanes: paclitaxel (Taxol®) and docetaxel (Taxotere®); Epothilones: ixabepilone (Ixempra®); Vinca alkaloids: vinblastine (Velban®), vincristine (Oncovin®), and vinorelbine (Navelbine®); Estramustine (Emcyt®); Prednisone; Methylprednisolone (Solumedrol®); Dexamethasone (Decadron®); L-asparaginase; bortezomib (Velcade®). Additional chemotherapy agents are listed, for example, in U.S. Patent Application Pub. No. 2008/0300165, which is incorporated herein by reference in its entirety.

Doses and administration protocols for chemotherapy drugs are well-known in the art. The skilled clinician can readily determine the proper dosing regimen to be used, based on factors including the chemotherapy agent(s) administered, type of cancer being treated, stage of the cancer, age and condition of the patient, patient size, location of the tumor, and the like.

Radiotherapy Agents

In one aspect of the present invention, AMD3100 is administered in combination with a radiotherapeutic agent. The radiotherapeutic agent may be any such agent having a therapeutic effect on one or more types of cancer. Many radiotherapeutic agents are currently known in the art. Types of radiotherapeutic drugs include, by way of non-limiting example, X-rays, gamma rays, and charged particles. In one embodiment, the radiotherapeutic agent is delivered by a machine outside of the body (external-beam radiation therapy). In a preferred embodiment, the radiotherapeutic agent is placed in the body near the tumor/cancer cells (brachytherapy) or is a systemic radiation therapy.

External-beam radiation therapy may be administered by any means. Exemplary, non-limiting types of external-beam radiation therapy include linear accelerator-administered radiation therapy, 3-dimensional conformal radiation therapy (3D-CRT), intensity-modulated radiation therapy (IMRT), image-guided radiation therapy (IGRT), tomotherapy, stereotactic radiosurgery, photon therapy, stereotactic body radiation therapy, proton beam therapy, and electron beam therapy.

Internal radiation therapy (brachytherapy) may be by any technique or agent. Exemplary, non-limiting types of internal radiation therapy include any radioactive agents that can be placed proximal to or within the tumor, such as Radium-226 (Ra-226), Cobalt-60 (Co-60), Cesium-137 (Cs-137), cesium-131, Iridium-192 (Ir-192), Gold-198 (Au-198), Iodine-125(I-125), palladium-103, yttrium-90, etc. Such agents may be administered by seeds, needles, or any other route of administration, and my be temporary or permanent.

Systemic radiation therapy may be by any technique or agent. Exemplary, non-limiting types of systemic radiation therapy include radioactive iodine, ibritumomab tiuxetan (Zevalin®), tositumomab and iodine I 131 tositumomab (Bexxar®), samarium-153-lexidronam (Quadramet®), strontium-89 chloride (Metastron®), metaiodobenzylguanidine, lutetium-177, yttrium-90, strontium-89, and the like.

In one embodiment, a radiosensitizing agent is also administered to the patient. Radiosensitizing agents increase the damaging effect of radiation on cancer cells.

Doses and administration protocols for radiotherapy agents are well-known in the art. The skilled clinician can readily determine the proper dosing regimen to be used, based on factors including the agent(s) administered, type of cancer being treated, stage of the cancer, location of the tumor, age and condition of the patient, patient size, and the like.

Anti-Cancer Vaccines

In one aspect of the present invention, AMD3100 is administered in combination with an anti-cancer vaccine (also called cancer vaccine). Anti-cancer vaccines are vaccines that either treat existing cancer or prevent development of a cancer by stimulating an immune reaction to kill the cancer cells. In a preferred embodiment, the anti-cancer vaccine treats existing cancer.

The anti-cancer vaccine may be any such vaccine having a therapeutic effect on one or more types of cancer. Many anti-cancer vaccines are currently known in the art. Such vaccines include, without limitation, dasiprotimut-T, Sipuleucel-T, talimogene laherparepvec, HSPPC-96 complex (Vitespen), L-BLP25, gp100 melanoma vaccine, and any other vaccine that stimulates an immune response to cancer cells when administered to a patient.

Cancers

Cancers or tumors that can be treated by the compounds and methods described herein include, but are not limited to: biliary tract cancer; brain cancer, including glioblastomas and medulloblastomas; breast cancer; cervical cancer; choriocarcinoma; colon cancer; endometrial cancer; esophageal cancer, gastric cancer; hematological neoplasms, including acute lymphocytic and myelogenous leukemia; multiple myeloma; AIDS associated leukemias and adult T-cell leukemia lymphoma; intraepithelial neoplasms, including Bowen's disease and Paget's disease; liver cancer (hepatocarcinoma); lung cancer; lymphomas, including Hodgkin's disease and lymphocytic lymphomas; neuroblastomas; oral cancer, including squamous cell carcinoma; ovarian cancer, including those arising from epithelial cells, stromal cells, germ cells and mesenchymal cells; pancreas cancer; prostate cancer; rectal cancer; sarcomas, including leiomyosarcoma, rhabdomyosarcoma, liposarcoma, fibrosarcoma and osteosarcoma; skin cancer, including melanoma, Kaposi's sarcoma, basocellular cancer and squamous cell cancer; testicular cancer, including germinal tumors (seminoma, non-seminoma[teratomas, choriocarcinomas]), stromal tumors and germ cell tumors; thyroid cancer, including thyroid adenocarcinoma and medullar carcinoma; and renal cancer including adenocarcinoma and Wilms tumor. In important embodiments, cancers or tumors escaping immune recognition include glioma, colon carcinoma, colorectal cancer, lymphoid cell-derived leukemia, choriocarcinoma, and melanoma.

In a preferred embodiment, the tumor is a solid tumor. In one embodiment, the tumor is a leukemia. In an especially preferred embodiment, the tumor over-expresses CXCL12. In one embodiment, tumor expression of CXCL12 can be evaluated prior to administration of a composition as described herein. For example, a patient having a tumor that is determined to express or over-express CXCL12 will be treated using a method and/or composition as described herein.

In one embodiment, the tumor is a brain tumor. It is contemplated that a brain tumor, e.g., an inoperable brain tumor, can be injected with a composition described herein. In one embodiment, AMD3100 is administered directly to a brain tumor via a catheter into a blood vessel within or proximal to the brain tumor. Further discussion of catheter or microcatheter administration is described below.

Dose and Administration

The compositions, as described herein, are administered in effective amounts. The effective amount will depend upon the mode of administration, the particular condition being treated and the desired outcome. It will also depend upon, as discussed above, the stage of the condition, the age and physical condition of the subject, the nature of concurrent therapy, if any, and like factors well known to the medical practitioner. For therapeutic applications, it is that amount sufficient to achieve a medically desirable result.

The agents described herein may be administered by any appropriate method. Dosage, treatment protocol, and routes of administration for anti-cancer agents, including chemotherapeutic agents, radiotherapeutic agents, and anti-cancer vaccines, as well as immunotherapy agents are known in the art and/or within the ability of a skilled clinician to determine, based on the type of treatment, type of cancer, etc.

Generally, the dose of the AMD3100 of the present invention is from about 5 mg/kg body weight per day to about 50 mg/kg per day, inclusive of all values and ranges therebetween, including endpoints. In one embodiment, the dose is from about 10 mg/kg to about 50 mg/kg per day inclusive of all values and ranges therebetween, including endpoints. In one embodiment, the dose is from about 10 mg/kg to about 40 mg/kg per day. In one embodiment, the dose is from about 10 mg/kg to about 30 mg/kg per day. In a preferred embodiment, the dose is from about 10 mg/kg to about 20 mg/kg per day. In one embodiment, the dose does not exceed about 50 mg per day.

In one embodiment, the dose of the AMD3100 is from about 50 mg/kg per week to about 350 mg/kg per week, inclusive of all values and ranges therebetween, including endpoints. In one embodiment, the dose of the AMD3100 is about 70 mg/kg per week. In one embodiment, the dose of the AMD3100 is about 80 mg/kg per week. In one embodiment, the dose of the AMD3100 is about 90 mg/kg per week. In one embodiment, the dose of the AMD3100 is about 100 mg/kg per week. In one embodiment, the dose of the AMD3100 is about 110 mg/kg per week. In one embodiment, the dose of the AMD3100 is about 120 mg/kg per week. In one embodiment, the dose of the AMD3100 is about 130 mg/kg per week. In one embodiment, the dose of the AMD3100 is about 140 mg/kg per week. In one embodiment, the dose of the AMD3100 is about 150 mg/kg per week. In one embodiment, the dose of the AMD3100 is about 160 mg/kg per week. In one embodiment, the dose of the AMD3100 is about 170 mg/kg per week. In one embodiment, the dose of the AMD3100 is about 180 mg/kg per week. In one embodiment, the dose of the AMD3100 is about 190 mg/kg per week. In one embodiment, the dose of the AMD3100 is about 200 mg/kg per week. In one embodiment, the dose of the AMD3100 is about 210 mg/kg per week. In one embodiment, the dose of the AMD3100 is about 220 mg/kg per week. In one embodiment, the dose of the AMD3100 is about 230 mg/kg per week. In one embodiment, the dose of the AMD3100 is about 240 mg/kg per week. In one embodiment, the dose of the AMD3100 is about 250 mg/kg per week. In one embodiment, the dose of the AMD3100 is about 260 mg/kg per week. In one embodiment, the dose of the AMD3100 is about 270 mg/kg per week. In one embodiment, the dose of the AMD3100 is about 280 mg/kg per week. In one embodiment, the dose of the AMD3100 is about 290 mg/kg per week. In one embodiment, the dose of the AMD3100 is about 300 mg/kg per week. In one embodiment, the dose of the AMD3100 is about 310 mg/kg per week. In one embodiment, the dose of the AMD3100 is about 320 mg/kg per week. In one embodiment, the dose of the AMD3100 is about 330 mg/kg per week. In one embodiment, the dose of the AMD3100 is about 340 mg/kg per week. In one embodiment, the dose of the AMD3100 is about 350 mg/kg per week.

In one aspect of the invention, the AMD3100 and the immunotherapy agent are administered sequentially. That is, the AMD3100 is administered for a period of time sufficient to have an anti-fugetactic effect, and the immunotherapy agent is subsequently administered. In one embodiment, an anti-cancer therapy is optionally administered.

In one aspect of the invention, administration of the AMD3100 is pulsatile. In one embodiment, an amount of AMD3100 is administered every 1 hour to every 24 hours, for example every 1 hour, 2 hours, 3 hours, 4 hours, 5 hours, 6 hours, 7 hours, 8 hours, 9 hours, 10 hours, 11 hours, 12 hours, 13 hours, 14 hours 15 hours, 16 hours, 17 hours, 18 hours, 19 hours, 20 hours, 21 hours, 22 hours, 23 hours, or 24 hours. In one embodiment, an amount of AMD3100 is administered every 1 day, 2 days, 3 days, 4 days, 5 days, 6 days, 7 days, 8 days, 9 days, 10 days, 11 days, 12 days, 13 days, or 14 days.

In one aspect of the invention, doses of the AMD3100 are administered in a pulsatile manner for a period of time sufficient to have an anti-fugetactic effect (e.g. to attenuate the fugetactic effect of the tumor cell). In one embodiment, the period of time is between about 1 day and about 14 days. For example, the period of time may be 1 day, 2 days, 3 days, 4 days, 5 days, 6 days, 7 days, 8 days, 9 days, 10 days and so on.

In one aspect of the invention, the immunotherapy agent is administered after the period of time of administration, e.g., 10 days. In one embodiment, the immunotherapy agent is administered during a period of time wherein the fugetactic effect of the cancer cells/tumor is attenuated by the AMD3100. The length of time and modes of administration of the immunotherapy agent will vary, depending on the immunotherapy agent used, type of tumor being treated, condition of the patient, and the like. Determination of such parameters is within the capability of the skilled clinician.

In one embodiment, administration of the AMD3100 for a period (e.g., 10 days) and the immunotherapy agent is alternated. For example, AMD3100 can be administered for a period of 10 days, followed by administration of the immunotherapy agent, followed by 10 days of AMD3100, etc. In a preferred embodiment, administration of the AMD3100 and the immunotherapy agent is alternated until the condition of the patient improves. Improvement includes, without limitation, reduction in size of the tumor and/or metastases thereof, elimination of the tumor and/or metastases thereof, remission of the cancer, and/or attenuation of at least one symptom of the cancer.

A variety of administration routes are available. The methods of the invention, generally speaking may be practiced using any mode of administration that is medically acceptable, meaning any mode that produces effective levels of the active compounds without causing clinically unacceptable adverse effects.

Modes of administration include oral, rectal, topical, nasal, interdermal, or parenteral routes. The term “parenteral” includes subcutaneous, intravenous, intramuscular, or infusion. Intravenous or intramuscular routes are not particularly suitable for long-term therapy and prophylaxis. They could, however, be preferred in emergency situations. Oral administration will be preferred for prophylactic treatment because of the convenience to the patient as well as the dosing schedule. When peptides are used therapeutically, in certain embodiments a desirable route of administration is by pulmonary aerosol. Techniques for preparing aerosol delivery systems containing peptides are well known to those of skill in the art. Generally, such systems should utilize components which will not significantly impair the biological properties of the antibodies, such as the paratope binding capacity (see, for example, Sciarra and Cutie, “Aerosols,” in Remington's Pharmaceutical Sciences, 18th edition, 1990, pp 1694-1712; incorporated by reference). Those of skill in the art can readily determine the various parameters and conditions for producing antibody or peptide aerosols without resort to undue experimentation.

Compositions suitable for oral administration may be presented as discrete units, such as capsules, tablets, lozenges, each containing a predetermined amount of the active agent(s). Other compositions include suspensions in aqueous liquids or non-aqueous liquids such as a syrup, elixir or an emulsion.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed 25 oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Lower doses will result from other forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of compounds.

In one embodiment, the AMD3100 is administered parenterally. In one embodiment, the AMD3100 is administered via microcatheter into a blood vessel proximal to a tumor. In one embodiment, the AMD3100 is administered via microcatheter into a blood vessel within a tumor. In one embodiment, the AMD3100 is administered subcutaneously. In one embodiment, the AMD3100 is administered intradermally.

Other delivery systems can include time-release, delayed release, or sustained release delivery systems. Such systems can avoid repeated administrations of the AMD3100, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono- di- and tri-glycerides; hydrogel release systems; sylastic systems; peptide based systems; wax coatings; compressed tablets using conventional binders and excipients; partially fused implants; and the like.

In one embodiment, the AMD3100 is administered in a time-release, delayed release or sustained release delivery system. In one embodiment, the time-release, delayed release or sustained release delivery system comprising the AMD3100 is inserted directly into the tumor. In one embodiment, the time-release, delayed release or sustained release delivery system comprising the AMD3100 is implanted in the patient proximal to the tumor. Additional implantable formulations are described, for example, in U.S. Patent App. Pub. No. 2008/0300165, which is incorporated herein by reference in its entirety.

In addition, important embodiments of the invention include pump-based hardware delivery systems, some of which are adapted for implantation. Such implantable pumps include controlled-release microchips. A preferred controlled-release microchip is described in Santini, J T Jr. et al., Nature, 1999, 397:335-338, the contents of which are expressly incorporated herein by reference.

When administered, the pharmaceutical preparations of the invention are applied in pharmaceutically-acceptable amounts and in pharmaceutically-acceptably compositions. Such preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

Methods of Treatment

The compositions as described herein can be used in methods of treating cancer, inhibiting metastasis of a tumor, slowing tumor growth, etc., in a patient in need thereof by administration of an aggregate unit dose of AMD3100. In a preferred embodiment, the aggregate unit dose of AMD3100 is administered in combination with a an anti-cancer agent, such as for example, a chemotherapy agent, a radiotherapeutic agent, an anti-cancer vaccine (also called cancer vaccine), etc. The administration as a combination can be done, for example, via co-administration or sequential administration of an anti-cancer therapy so as to improve the efficacy of the therapy.

In one embodiment, the anti-cancer therapy or agent is administered after the period of time of administration of the aggregate unit dose of the AMD3100. In one embodiment, the immunotherapy agent is administered during a period of time when the AMD3100 is attenuated.

Kit of Parts

This invention further relates to a kit of parts comprising AMD3100 and at least one immunotherapy agent as described herein. In one embodiment, the kit of parts comprises a first container comprising AMD3100 and a second container comprising an immunotherapy agent. In one embodiment, the kit of parts further comprises instructions in a readable medium for dosing and/or administration of the AMD3100 and immunotherapy agent.

The term “readable medium” as used herein refers to a representation of data that can be read, for example, by a human or by a machine. Non-limiting examples of human-readable formats include pamphlets, inserts, or other written forms. Non-limiting examples of machine-readable formats include any mechanism that provides (i.e., stores and/or transmits) information in a form readable by a machine (e.g., a computer, tablet, and/or smartphone). For example, a machine-readable medium includes read-only memory (ROM); random access memory (RAM); magnetic disk storage media; optical storage media; and flash memory devices. In one embodiment, the machine-readable medium is a CD-ROM. In one embodiment, the machine-readable medium is a USB drive. In one embodiment, the machine-readable medium is a Quick Response Code (QR Code) or other matrix barcode.

### EXAMPLES

The following examples are for illustrative purposes only and should not be interpreted as limitations of the claimed invention. There are a variety of alternative techniques and procedures available to those of skill in the art which would similarly permit one to successfully perform the intended invention.

### Example 1

Mice are injected with tumor cells (subcutaneous injection) from a tumor that expresses high levels of CXCL12 and a tumor allowed to develop. Once the tumor has formed, the mice are injected (subcutaneous in the same flank as the tumor) with AMD3100 or vehicle, once a day for 5 days.

One to three days after the final dose of AMD3100, mice are injected via intraperitoneal injection with a therapeutically effective amount of cisplatin 18 hours prior to assay of tumor growth. Tumor growth in mice is delayed by cisplatin treatment, but resumes soon after the treatment is discontinued in mice that were not administered AMD3100. It is contemplated that treatment with AMD3100 prior to treatment with cisplatin will have a synergistic effect, such that the co-treatment results in a delay in tumor growth that is longer than cisplatin alone.

What is claimed is:

1. A pharmaceutical product comprising an aggregate unit dose of AMD3100 of about 50 to 350 mg/kg for administration over a 7 to 14 day period of time.

2. A kit of parts comprising a first container comprising an aggregate unit dose of AMD3100 from about 50-350 mg/kg, and a second container comprising an anti-cancer agent or therapy.

3. The kit of parts of claim 2, further comprising a readable medium indicating the complete administration of the aggregate unit dose of AMD3100 prior to the administration of the anti-cancer agent.
